(19) United States
(12) Patent Application Publication
(10) Pub. No.: US 2023/0348567 A1
(43) Pub. Date: Nov. 2, 2023

(54) COLLAGEN WITH SELECTIVE CHARACTERISTICS, COLLAGEN PRODUCTS CONTAINING SAME AND METHODS FOR PRODUCING SAME

(71) Applicant: COLLAGEN MATRIX, INC., Oakland, NJ (US)

(72) Inventors: Natsuyo Shishido Lee, Basking Ridge, NJ (US); Daniel Ammon, Webster, NY (US); Hui-Chen Chen, Wayne, NJ (US)

(73) Assignee: COLLAGEN MATRIX, INC., Oakland, NJ (US)

(21) Appl. No.: 18/025,963

(22) PCT Filed: Sep. 9, 2021
(86) PCT No.: PCT/US2021/049646

§ 371 Date: Mar. 13, 2023

Related U.S. Application Data
(60) Provisional application No. 63/149,068, filed on Feb. 12, 2021.
(60) Provisional application No. 63/093,554, filed on Oct. 19, 2020.
(60) Provisional application No. 63/079,187, filed on Sep. 16, 2020.

(51) Int. Cl. C07K 1/107; C07K 14/78

(52) U.S. Cl. CPC C07K 14/78; C07K 1/1075

(57) ABSTRACT

Disclosed are methods for forming targeted collagen products having an amplified desired characteristic, including the step of adding peptides exhibiting a desired characteristic to collagen to form the targeted collagen product. Further disclosed are methods for forming a targeted collagen product lacking an undesired characteristic, including the step of subtracting peptides exhibiting the undesired characteristic from collagen to form the targeted collagen product. Also disclosed are targeted collagen products formed by the disclosed methods.

### RELATED APPLICATIONS

This application claims priority to (1) U.S. Provisional Pat. Application No. 63/079,187, filed Sep. 16, 2020; (2) U.S. Provisional Pat. Application No. 63/093,554, filed Oct. 19, 2020; and (3) U.S. Provisional Pat. Application No. 63/149,068, filed Feb. 12, 2021, all three of which are incorporated herein by reference in their entireties.

### FIELD OF THE INVENTION

Embodiments of the invention pertain to customizable collagen intended for application to various tissues for diverse purposes, including biological tissue repair. More particularly, embodiments of the invention relate to methods for amplifying or turning down certain characteristics of collagen for targeted uses, such as for soft tissue repair applications, or bone repair applications. Embodiments of the invention also relate to engineered collagen constructs, including layered targeted collagen products/constructs, methods for making same, methods using 3D printing of biomolecules including the customizable collagen, and collagen constructs formed by such methods.

### BACKGROUND OF THE INVENTION

Collagen is a naturally occurring protein found in humans and animals. Collagen tissue is often procured from a donor, and used in a wide variety of medical applications ranging from cosmetic surgery to bone repair to wound healing. Prokaryote collagen can also be derived from genetically engineered microorganisms as well as from genetically engineered plants

Collagen may be treated and/or processed in a number of different ways such as being augmented, reconstituted, concentrated, cross-linked, combined with other biological substances, and so on. As such, various collagen products may be produced for diverse medical applications.

Some embodiments of the present invention relate generally to rapid prototyping systems, specifically, 3D printing systems for making collagen based medical and dental devices such as, for example, dental bone grafting, dental membranes, stents, punctal plugs, ocular collagen onlays and inlays, contact lenses, orthopedic bone application, Spine application, nerve applications and skin applications. More particularly, the invention relates to the use of ink-jet, fused deposition modeling (FDM), selective laser sintering (SLS), stereolithography (SLA), digital light processing (DLP), Bio-printing or their combinations to build-up the medical devices as three-dimensional objects from many material systems and novel resin systems of this invention. Ink-jet printing system dispenses materials through ink-jet printing head to form 3D object, which harden by cooling, polymerization, and light irradiation. FDM extrudes thermoplastic materials throughout nozzle to build 3D object. SLS uses laser as power source to sinter powdered materials to form solid objects. SLA using laser beam traces out the shape of each layer and hardens the photosensitive resin in a vat (reservoir or bath). DLP system builds three-dimensional objects by using the Digital Light Processor (DLP) projector to project sequential voxel planes into liquid resin, which then caused the liquid resin to cure. Bioprinting is a layer-by-layer process is which a biological matrix is printed either with or without cells. The object then can act as a matrix or scaffold to grow cellularized tissue. In general, rapid prototyping refers to a conventional manufacturing process used to make parts, wherein the part is built on a layer-by-layer basis using layers of hardening material. Per this technology, the part to be manufactured is considered a series of discrete cross-sectional regions which, when combined, make-up a three-dimensional structure. The building-up of a part layer-by-layer is very different than conventional machining technologies, where metal or plastic pieces are cut and drilled to a desired shape. In rapid prototyping technology, the parts are produced directly from computer-aided design (CAD) or other digital images. Software is used to slice the digital image into thin cross-sectional layers. Then, the part is constructed by placing layers of plastic or other hardening material on top of each other. There are many different techniques that can be used to combine the layers of structural material. A final curing step may be required to fully cure the layers of material for some of the techniques. The application of sealer may be needed to form a dense 3D object for some of the techniques, such as inkjet printing of a powder bed or FDM. Additional milling may be added to some of the techniques.

Ink-jet printing technology is a rapid prototyping method that can be used to fabricate the three-dimensional object. In one well known ink-jet printing method that was developed at Massachusetts Institute of Technology, as described in Sachs et al., U.S. Pat. No. 5,204,055 (incorporated by reference herein in its entirety), printer heads are used to discharge a binder material onto a layer of powder particulate in a powder bed. The powdered layer corresponds to a digitally superposed section of the object that will be produced. The binder causes the powder particles to fuse together in selected areas. This results in a fused cross-sectional segment of the object being formed on the platform. The steps are repeated for each new layer until the desired object is achieved. In a final step, a laser beam scans the object causing the powdered layers to sinter and fuse together if needed. In another ink-jet printing process, as described in Sanders, U.S. Pat. Nos. 5,506,607 and 5,740,051, a low-melting thermoplastic material is dispensed through one ink-jet printing head to form a three-dimensional object. A second ink-jet printer head dispenses wax material or other supporting material to form supports for the three-dimensional object. After the object has been produced, the wax supports are removed, and the object is finished as needed. MultiJet printers, such as, the high-quality PolyJet and MultiJet 3D printing processes use a UV light to crosslink a photopolymer. However, rather than scanning a laser to cure layers, a printer jet sprays tiny droplets of the photopolymer (similar to ink in an inkjet printer) in the shape of the first layer. The UV lamp attached to the printer head crosslinks the polymer and locks the shape of the layer in place. The build platform then descends by one layer thickness, and more material is deposited directly onto the previous layer. Triple-jetting technology (PolyJet) used in Stratasys Objet 500 Connex3, is the most advanced method of PolyJet 3D printing. This technology performs precise printing with three materials and thus makes three-color mixing possible.

Fused deposition modeling (FDM) technology was developed and implemented at first time by Scott Crump, Stratasys Ltd. founder, in 1980s. What is good about this technology that all parts printed with FDM can go in high-performance and engineering-grade thermoplastic. FDM is the only 3D printing technology that builds parts with production-grade thermoplastics, so things printed are of excellent mechanical, thermal and chemical qualities. 3D printing machines that use FDM Technology build objects layer by layer from the bottom up by heating and extruding thermoplastic filament. Along to thermoplastic a printer can extrude support materials as well. Then the printer heats thermoplastic till its melting point and extrudes it throughout nozzle to a build platform. To support upper layer the printer may place underneath special material that can be dissolved after printing is completed. When the thin layer of plastic binds to the layer beneath it, it cools down and hardens. Once the layer is finished, the base is lowered to start building of the next layer. This technology is considered simple-to-use and environment-friendly. Different kind of thermoplastics can be used to print dental objects.

Selective Laser Sintering (SLS) is a technique that uses laser as power source to form solid 3D objects. This technique was developed by Carl Deckard, a student of Texas University, and his professor Joe Beaman in 1980s. The main difference between SLS and SLA is that it uses powdered material in the vat instead of liquid resin as stereolithography does. Unlike some other additive manufacturing processes, such as stereolithography (SLA) and fused deposition modeling (FDM), SLS doesn’t need to use any support structures as the object being printed is constantly surrounded by unsintered powder. Due to wide variety of materials that can be used with this type of 3D printer the technology is very popular for 3D printing customized products. SLS requires the use of high-powered lasers, which makes the printer to be very expensive. Extensive surface finishing is required for dental objects made with this process.

SLA 3D printing method was patented by Charles Hull, co-founder of 3D Systems, Inc. in 1986, which converts liquid plastic into solid 3D objects. SLA 3D printers work with excess of liquid resin that hardens and forms into solid object by irradiation. Parts built usually have smooth surfaces but its quality varies depending on the quality of SLA machine used. After plastic hardens a platform of the printer drops down (top-down printer) or moves up (bottom up printer) in the tank a fraction of a millimeter and laser-forms the next layer until printing is completed. Once all layers are printed the object is rinsed with a solvent and then placed in a post-cure oven to finish processing.

Digital Light Processing is another 3D Printing process very similar to stereolithography. The DLP technology was created in 1987 by Larry Hornbeck of Texas Instruments and became very popular in Projectors production. It uses digital micro mirrors laid out on a semiconductor chip. 3D inkjet, DLP and SLA all works with photopolymers. The difference between SLA and DLP processes is a different light source. DLP method projects sequential voxel planes into liquid resin, which then caused the liquid resin to cure. The material used for printing is liquid resin that is placed in the transparent resin container. The resin hardens quickly when affected by irradiation of light. The printing speed is impressive, especially with Carbon3D’s CLIP (Continuous Liquid Interface Production) technology. The layer of hardened material can be created with such printer in a few seconds. When the layer is finished, itis moved up and the next layer is started to be worked on. CLIP technology balances light and oxygen to eliminate the mechanical steps and layers that are the standard DLP process step and allow the production of commercial quality objects at high speed.

BioPrinting, containing one or more of cells, matrix, and nutrients known as bioinks are placed in a printer cartridge and deposited using the patients’ medical scans. When a bioprinted pre-tissue is transferred to an incubator, this cell-based pre-tissue matures into a tissue. Also, a matrix may be printed without cell and then populated with cell, in-vivo or ex-viso.

3D bioprinting for fabricating biological constructs typically involves dispensing cells onto a biocompatible scaffold using a successive layer-by-layer approach to generate tissue-like three-dimensional structures. Given that every tissue in the body is naturally composed of different cell types, many technologies for printing these cells vary in their ability to ensure stability and viability of the cells during the manufacturing process. Some of the methods that are used for 3D bioprinting of cell some of the printing techniques mentioned above as well as extrusion printing into a support gel.

An aspect of the invention pertains to the utilization of 3D printing to digitally process dental and medical devices as well as ECM scaffolds and cellular scaffolds primarily composed of collagen, modified collagen and/or collagen-based peptides. To produce polymerizable peptides, collagen is digested with an enzyme, then the peptides are modified with functional groups that can be polymerized with radiation. These modified peptides are then formulated with initiator(s), crosslinker(s), solvents and/or other additives to create the desired design inputs for a particular dental or medical application. This formulation can then be 3D printed. In another process, collagen is digested to create peptides, other peptides are added or subtracted to generate customized desired design inputs for a particular dental or medial application. Then these newly formulated peptides are modified with functional groups that can be polymerized with radiation. These modified peptides are then formulated with initiator(s), crosslinker(s), solvents and/or other additives to create the desired design inputs for a particular dental or medical application. This formulation then can be 3D printed. Another example involves extruding collagen that has been modified so that it is soluble in a solvent and optionally modified with functional chemistry so that an energy driven, post-process can be carried out. The extrudable collagen can also contain other collagen-based peptides, such as collagen mimetic peptides (“CMPs”), and collagen-hybridizing peptides (“CHPs”), to amplify collagen biological processes. The extrudable collagen can also contain other bioactive based peptides and growth factors to enhance the healing process.

3D printing is frequently called “rapid prototyping”. The present invention includes methods for utilizing 3D printing to make final manufactured products. Some embodiments of the present invention are directed toward the amplification of collagen by concentrating, diluting, adding or subtracting CMPs and CHPs in a dental or medical device. The device can be produced by molding or 3D printing.

### SUMMARY OF THE INVENTION

Provided herein are methods for forming a targeted collagen product having amplified desired characteristics or reduced undesired characteristics.

In one exemplary embodiment, a method for forming a targeted collagen product having an amplified desired characteristic includes the step of adding peptides exhibiting a desired characteristic to collagen to form the targeted collagen product.

In another exemplary embodiment, a method for forming a targeted collagen product lacking an undesired characteristic includes the step of subtracting peptides exhibiting the undesired characteristic from collagen to form the targeted collagen product.

Further aspects of the invention include the following:

1. A method for forming a targeted collagen product having an amplified desired characteristic, comprising: procuring collagen; digesting the collagen to derive peptides therefrom; testing the peptides for a desired characteristic; isolating the peptides exhibiting the desired characteristic; and adding the peptides exhibiting the desired characteristic to a mixture to reconstitute collagen into the targeted collagen product.

2. The method of aspect 1 above, wherein the procuring step includes deriving collagen from eukaryotes or genetically modified prokaryotes.

3. The method of aspect 1 above, wherein the digesting step includes subjecting the procured collagen to enzymes.

4. The method of aspect 3 above, wherein the enzymes are selected from the group consisting of collagenase, pepsin, papain and pancreatin.

5. The method of aspect 1 above, wherein the desired characteristic of the isolated peptides has an application selected from the group consisting of wound healing, osteogenesis, blood clotting, organ scaffolds, dura repair, nerve repair, ocular repair, epithelial repair, cardio-vascular repair, endothelial repair, breast repair, antioxidants, antibacterial, anti-inflammatory activity, and for the delivery of drugs.

6a. The method of aspect 1 above, wherein the targeted collagen product is a device.

6b. The method of aspect 1 above, wherein the adding step includes adding one or more amplification peptides to the mixture to reconstitute collagen.

7. The method of aspect 6b above, wherein the adding step includes covalently linking the amplification peptide(s) into the reconstituted collagen.

8. The method of aspect 6b above, wherein the adding step includes non-covalently linking the amplification peptide(s) into the reconstituted collagen.

9. A method of aspect 8 above, wherein the non-covalently linked amplification peptide(s) are released over a period of time.

10. A method for forming a targeted collagen product lacking an undesired characteristic, comprising: procuring collagen; digesting the collagen to derive peptides therefrom; testing the peptides for the undesired characteristic; isolating the peptides exhibiting the undesired characteristic; and subtracting the peptides exhibiting the undesired characteristic from a collagen mixture; and reconstituting the collagen mixture to form the targeted collagen product.

11. A method for forming a targeted collagen product having an amplified desired characteristic, comprising: procuring collagen; digesting the collagen to derive peptides therefrom; testing the peptides for a desired characteristic; isolating the peptides exhibiting the desired characteristic; and adding the peptides exhibiting the desired characteristic to intact collagen to form the targeted collagen product.

12. The method of aspect 11 above, wherein the procuring step includes deriving collagen from eukaryotes or genetically modified prokaryotes.

13. The method of aspect 11 above, wherein the digesting step includes subjecting the procured collagen to enzymes.

14. The method of aspect 13 above, wherein the enzymes are selected from the group consisting of collagenase and pepsin,

15. The method of aspect 11 above, wherein the desired characteristic of the isolated peptides has an application selected from the group consisting of wound healing, osteogenesis, blood clotting, organ scaffolds, dura repair, nerve repair, ocular repair, epithelial repair, breast repair, and cardio-vascular repair, endothelial repair

16. The method of aspect 11 above, wherein the adding step includes adding one or more amplification peptides to the mixture to reconstitute collagen.

17. The method of aspect 16 above, wherein the adding step includes covalently linking the amplification peptide(s) into the intact collagen.

18. The method of aspect 16 above, wherein the adding step includes non-covalently linking the amplification peptide(s) into the intact collagen.

19. A method of aspect 18 above, wherein the non-covalently linked amplification peptide(s) are released over a period of time.

20. A method for forming a targeted collagen product lacking an undesired characteristic, comprising: procuring collagen; digesting the collagen to derive peptides therefrom; testing the peptides for the undesired characteristic; isolating the peptides exhibiting the undesired characteristic; and subtracting the peptides exhibiting the undesired characteristic from intact collagen to form the targeted collagen product.

21. A method for forming a targeted collagen product having an amplified or de-amplified desired characteristic, comprising: genetically engineering cells to produce modified collagen.

22. The method of aspect 21 above, wherein the cells are selected from the group consisting of a prokaryotic cell and a eukaryotic cell and plant cells.

23. A method for forming a targeted collagen product having an amplified desired characteristic, comprising: procuring collagen; digesting the collagen to derive peptides therefrom; testing the peptides for a desired characteristic; isolating the peptides exhibiting the desired characteristic; and adding the peptides exhibiting the desired characteristic to a mixture to reconstitute collagen into the targeted collagen product so that the reconstituted protein can control the release profile of bioactive proteins or peptides.

24. A method for forming a targeted collagen product lacking an undesired characteristic, comprising: procuring collagen; digesting the collagen to derive peptides therefrom; testing the peptides for an undesired characteristic; isolating the peptides exhibiting the undesired characteristic; and subtracting the peptides exhibiting the undesired characteristic from a collagen mixture; and reconstituting the collagen mixture to form the targeted collagen product so that the reconstituted protein can control the release profile of bioactive proteins or peptides.

25. A method for forming a targeted collagen product having an amplified desired characteristic, comprising: adding peptides exhibiting a desired characteristic to a mixture to reconstitute collagen into the targeted collagen product.

26. The method of aspect 25 above, further comprising the step of procuring collagen.

27. The method of aspect 25 above, further comprising the step of digesting collagen to derive peptides therefrom.

28. The method of aspect 25 above, further comprising the step of testing peptides for the desired characteristic.

29. The method of aspect 25 above, further comprising the step of isolating peptides exhibiting the desired characteristic.

30. A method for forming a targeted collagen product lacking an undesired characteristic, comprising: subtracting peptides exhibiting the undesired characteristic from a collagen mixture to form the targeted collagen product.

31. The method of aspect 30 above, further comprising the step of procuring collagen.

32. The method of aspect 30 above, further comprising the step of digesting collagen to derive peptides therefrom.

33. The method of aspect 30 above, further comprising the step of testing peptides for the undesired characteristic.

34. The method of aspect 30 above, further comprising the step of isolating peptides exhibiting the undesired characteristic.

35. The method of aspect 30 above, further comprising the step of reconstituting the collagen mixture.

36. A method for forming a layered targeted collagen product having at least one selected characteristic that has been amplified, comprising: forming a first targeted collagen product having peptides exhibiting the at least one selected amplified characteristic; preparing a first collagen layer including the first targeted collagen product; and overlaying the first collagen layer onto a second layer.

37. The method of aspect 36 above, the preparing step includes adding the first targeted collagen product to a collagen layer to form the first collagen layer.

38. The method of aspect 36 above, wherein the preparing step includes forming the first targeted collagen product as the first collagen layer.

39. The method of aspect 36 above, wherein the second layer is a second collagen layer.

40. The method of aspect 39 above, wherein the second collagen layer includes a second targeted collagen product.

41. The method of aspect 40 above, wherein the at least one selected amplified characteristic includes a first selected amplified characteristic in the first targeted collagen product and first collagen layer, and a second selected amplified characteristic in the second targeted collagen product and second collagen layer; and wherein the first selected amplified characteristic is different from the second selected amplified characteristic.

42. The method of aspect 36 above, wherein the second layer is a substrate.

43. The method of aspect 42 above, wherein the substrate does not include collagen.

44. A layered targeted collagen product, comprising: a first collagen layer having a first targeted collagen product including a first amplified characteristic; and a second collagen layer overlaying the first collagen layer and having a second targeted collagen product including a second amplified characteristic; wherein the first amplified characteristic is different from the second amplified characteristic.

45. The product of aspect 44 above, wherein the first amplified characteristic and second amplified characteristic have applications selected from the group consisting of wound healing, osteogenesis, blood clotting, organ scaffolds, dura repair, nerve repair, ocular repair, epithelial repair, breast repair, and cardio-vascular repair, endothelial repair.

46. A layered targeted collagen product, comprising: a substrate and a collagen layer overlaying the substrate and having a targeted collagen product including at least one amplified characteristic.

47. The product of aspect 46 above, wherein the substrate includes unmodified collagen.

48. A method for forming a layered targeted collagen product lacking at least one selected undesired characteristic, comprising: forming a first targeted collagen product by subtracting peptides exhibiting the at least one selected undesired characteristic from collagen; preparing a first collagen layer including the first targeted collagen product; and overlaying the first collagen layer onto a second layer.

49. The method of aspect 48 above, wherein the preparing step includes adding the first targeted collagen product to a collagen layer to form the first collagen layer.

50. The method of aspect 48 above, wherein the preparing step includes forming the first targeted collagen product as the first collagen layer.

51. The method of aspect 48 above, wherein the second layer is a second collagen layer.

52. The method of aspect 51 above, wherein the second collagen layer includes a second targeted collagen product.

53. The method of aspect 52 above, wherein the at least one selected undesired characteristic includes a first selected undesired characteristic in the first targeted collagen product and first collagen layer, and a second selected undesired characteristic in the second targeted collagen product and second collagen layer; and wherein the first selected undesired characteristic is different from the second selected undesired characteristic.

54. The method of aspect 48 above, wherein the second layer is a substrate.

55. The method of aspect 54 above, wherein the substrate does not include collagen.

56. A layered targeted collagen product, comprising: a first collagen layer having a first targeted collagen product lacking a first selected undesired characteristic; and a second collagen layer overlaying the first collagen layer and having a second targeted collagen product lacking a second selected undesired characteristic; wherein the first selected undesired characteristic is different from the second selected undesired characteristic.

57. The product of aspect 56 above, wherein the first selected undesired characteristic and second selected undesired characteristic have applications selected from the group consisting of wound healing, osteogenesis, blood clotting, organ scaffolds, dura repair, nerve repair, ocular repair, epithelial repair, breast repair, and cardio-vascular repair, endothelial repair.

58. A layered targeted collagen product, comprising: a substrate and a collagen layer overlaying the substrate and having a targeted collagen product lacking at least one selected undesired characteristic.

59. The product of aspect 58 above, wherein the substrate includes unmodified collagen.

60. A method for forming a targeted collagen product having an amplified desired characteristic, comprising: adding collagen-based peptides exhibiting the desired characteristic to a collagen mixture containing collagen; and 3D printing the collagen mixture to form the targeted collagen product.

61. The method of aspect 60 above, wherein the collagen mixture includes cells and growth media.

62. The method of aspect 60 above, wherein the 3D printing step includes an operation selected from the group consisting of DLP, SLA, extrusion, bio-printing, and ink-jet printing.

63. The method of aspect 60 above, further comprising the step of post-processing the targeted collagen product.

64. The method of aspect 63 above, wherein the post-processing step includes adding cells to the targeted collagen product.

65. The method of aspect 63 above, wherein the post-processing step includes adding growth media to the targeted collagen product.

66. The method of aspect 63 above, wherein the post-processing step includes adding growth factors to the targeted collagen product.

67. The method of aspect 63 above, wherein the post-processing step includes extracting residual molecules from the targeted collagen product.

68. The method of aspect 63 above, wherein the post-processing step includes UV curing the targeted collagen product.

69. The method of aspect 63 above, wherein the post-processing step includes heat treating the targeted collagen product.

70. The method of aspect 63 above, wherein the post-processing step includes crosslinking the targeted collagen product.

71. The method of aspect 60 above, wherein the desired characteristic of the isolated peptides has an application selected from the group consisting of wound healing, osteogenesis, blood clotting, organ scaffolds, dura repair, nerve repair, ocular repair, epithelial repair, cardio-vascular repair, endothelial repair, breast repair, antioxidants, antibacterial, anti-inflammatory activity, and for the delivery of drugs.

72. The method of aspect 60 above, wherein the adding step includes adding one or more amplification peptides to the collagen mixture.

73. The method of aspect 72 above, wherein the adding step includes covalently linking the one or more amplification peptides to the collagen mixture.

74. The method of aspect 72 above, wherein the adding step includes non-covalently linking the one or more amplification peptides to the collagen mixture.

75. The method of aspect 74 above, wherein the one or more non-covalently linked amplification peptides are released over a period of time.

76. The method of aspect 60 above, further comprising the step of modifying the collagen of the collagen mixture.

77. The method of aspect 76 above, wherein the modifying step is performed with one or more from the group consisting of free radicals, condensation reaction and ring opening polymerization groups.

78. The method of aspect 60 above, wherein the collagen of the collagen mixture is soluble collagen.

79. The method of aspect 60 above, wherein the collagen of the collagen mixture is insoluble collagen.

80. The method of aspect 60 above, wherein the wherein the adding step includes adding one or more minerals to the collagen mixture.

81. The method of aspect 80 above, wherein the at least one mineral is hydroxyapatite or a hydroxyapatite-like substance.

82. A method for forming a targeted collagen product lacking an undesired characteristic, comprising: subtracting collagen-based peptides exhibiting the undesired characteristic from a collagen mixture containing collagen; and 3D printing the collagen mixture to form the targeted collagen product.

83. The method of aspect 82 above, wherein the 3D printing step includes an operation selected from the group consisting of DLP, SLA, extrusion, bio-printing, and ink-jet printing.

84. The method of aspect 82 above, further comprising the step of post-processing the targeted collagen product.

85. The method of aspect 84 above, wherein the post-processing step includes adding cells to the targeted collagen product.

86. The method of aspect 84 above, wherein the post-processing step includes adding growth media to the targeted collagen product.

87. The method of aspect 84 above, wherein the post-processing step includes adding growth factors to the targeted collagen product.

88. The method of aspect 84 above, wherein the post-processing step includes extracting residual molecules from the targeted collagen product.

89. The method of aspect 84 above, wherein the post-processing step includes UV curing the targeted collagen product.

90. The method of aspect 84 above, wherein the post-processing step includes heat treating the targeted collagen product.

91. The method of aspect 84 above, wherein the post-processing step includes crosslinking the targeted collagen product.

92. The method of aspect 82 above, further comprising the step of modifying the collagen of the collagen mixture.

93. The method of aspect 92 above, wherein the modifying step is performed with one or more from the group consisting of free radicals, condensation reaction and ring opening polymerization groups.

94. The method of aspect 82 above, wherein the collagen of the collagen mixture is soluble collagen.

95. The method of aspect 82 above, wherein the collagen of the collagen mixture is insoluble collagen.

96. The method of aspect 94 above, wherein the at least one mineral is hydroxyapatite or a hydroxyapatite-like substance.

97. A method for forming a targeted collagen product having an amplified desired characteristic, comprising: adding collagen-based peptides exhibiting the desired characteristic to collagen; and 3D printing the collagen to form the targeted collagen product.

98. A method for forming a targeted collagen product lacking an undesired characteristic, comprising: subtracting the peptides exhibiting the undesired characteristic from collagen; and 3D printing the collagen to form the targeted collagen product.

99. A method for forming a targeted collagen product having an amplified desired characteristic, comprising: genetically engineering cells to produce modified collagen; and 3D printing the modified collagen to form the targeted collagen product.

100. The method of aspect 99 above, wherein the cells are selected from the group consisting of prokaryotic cells, eukaryotic cells and plant cells.

101. The method of any of aspects 60, 82, 97 or 98 above, further comprising the step of procuring collagen.

102. The method of any of aspects 60, 82, 97 or 98 above, further comprising the step of digesting collagen to derive the collagen-based peptides therefrom.

103. The method of aspect 102 above, further comprising the step of testing the collagen-based peptides for the desired characteristic or the undesired characteristic.

104. The method of any of aspects 60, 82, 97 or 98 above, further comprising the step of isolating peptides exhibiting the desired characteristic or the undesired characteristic.

105. The method of any of aspects 60, 82, 97 or 98 above, further comprising the step of adding one or more growth factors to the collagen mixture.

106. A method for forming a layered targeted collagen product having at least one selected characteristic that has been amplified, comprising: forming a first targeted collagen product having peptides exhibiting the at least one selected amplified characteristic; preparing a first collagen layer including the first targeted collagen product; and overlaying the first collagen layer onto a second layer.

107. The method of aspect 106 above, the preparing step includes adding the first targeted collagen product to a collagen layer to form the first collagen layer.

108. The method of aspect 106 above, wherein the preparing step includes forming the first targeted collagen product as the first collagen layer.

109. The method of aspect 106 above, wherein the second layer is a second collagen layer that includes a second targeted collagen product.

110. The method of aspect 109 above, wherein the at least one selected amplified characteristic includes a first selected amplified characteristic in the first targeted collagen product and first collagen layer, and a second selected amplified characteristic in the second targeted collagen product and second collagen layer; and wherein the first selected amplified characteristic is different from the second selected amplified characteristic.

111. The method of aspect 106 above, wherein the second layer is a substrate that does not include collagen.

112. A layered targeted collagen product, comprising: a first collagen layer having a first targeted collagen product including a first amplified characteristic; and a second layer overlaying the first collagen layer.

113. The layered targeted collagen product of aspect 112 above, wherein the second layer is a second collagen layer having a second targeted collagen product including a second amplified characteristic; wherein the first amplified characteristic is different from the second amplified characteristic.

114. The layered targeted collagen product of aspect 113 above, wherein the first amplified characteristic and second amplified characteristic have applications selected from the group consisting of wound healing, osteogenesis, blood clotting, organ scaffolds, dura repair, nerve repair, ocular repair, epithelial repair, breast repair, and cardio-vascular repair, endothelial repair.

115. The layered targeted collagen product of aspect 112 above, wherein the second layer is a substrate.

116. The layered targeted collagen product of aspect 115 above, wherein the substrate includes unmodified collagen.

117. A method for forming a layered targeted collagen product lacking at least one selected undesired characteristic, comprising: forming a first targeted collagen product by subtracting peptides exhibiting the at least one selected undesired characteristic from collagen; preparing a first collagen layer including the first targeted collagen product; and overlaying the first collagen layer onto a second layer.

118. The method of aspect 117 above, wherein the preparing step includes adding the first targeted collagen product to a collagen layer to form the first collagen layer.

119. The method of aspect 117 above, wherein the preparing step includes forming the first targeted collagen product as the first collagen layer.

120. The method of aspect 117 above, wherein the second layer is a second collagen layer that includes a second targeted collagen product.

121. The method of aspect 120 above, wherein the at least one selected undesired characteristic includes a first selected undesired characteristic in the first targeted collagen product and first collagen layer, and a second selected undesired characteristic in the second targeted collagen product and second collagen layer; and wherein the first selected undesired characteristic is different from the second selected undesired characteristic.

122. The method of aspect 117 above, wherein the second layer is a substrate that does not include collagen.

123. A layered targeted collagen product, comprising: a first collagen layer having a first targeted collagen product lacking a first selected undesired characteristic; and a second layer overlaying the first collagen layer.

124. The layered targeted collagen product of aspect 123 above, wherein the second layer is a second collagen layer having a second targeted collagen product lacking a second selected undesired characteristic; wherein the first selected undesired characteristic is different from the second selected undesired characteristic.

125. The product of aspect 124 above, wherein the first selected undesired characteristic and second selected undesired characteristic have applications selected from the group consisting of wound healing, osteogenesis, blood clotting, organ scaffolds, dura repair, nerve repair, ocular repair, epithelial repair, breast repair, and cardio-vascular repair, endothelial repair.

126. The layered targeted collagen product of aspect 123 above, wherein the second layer is a substrate.

127. The layered targeted collagen product of aspect 126 above, wherein the substrate includes unmodified collagen.

128. A method for forming a targeted collagen product having an amplified desired characteristic, comprising: adding peptides exhibiting a desired characteristic to collagen to form the targeted collagen product.

129. The method of aspect 128 above, further comprising the step of digesting the collagen to derive the peptides exhibiting the desired characteristic therefrom.

130. The method of aspect 129 above, further comprising the step of procuring the collagen for the digesting step, and wherein the procuring step includes deriving collagen from eukaryotes or genetically modified prokaryotes.

131. The method of aspect 129 above, further comprising the step of testing the peptides for the desired characteristic.

132. The method of aspect 131 above, further comprising the step of isolating the peptides exhibiting the desired characteristic.

133. The method of aspect 128 above, wherein the adding step includes adding the peptides exhibiting the desired characteristic to a mixture to reconstitute collagen into the targeted collagen product.

134. The method of aspect 133 above, wherein the adding step is performed so that the reconstituted collagen can control the release profile of the peptide exhibiting the desired characteristic.

135. The method of aspect 128 above, wherein the adding step includes adding the peptides exhibiting the desired characteristic to intact collagen.

136. The method of aspect 128 above, wherein the desired characteristic of the peptides has an application selected from the group consisting of wound healing, osteogenesis, blood clotting, organ scaffolds, dura repair, nerve repair, ocular repair, epithelial repair, breast repair, and cardio-vascular repair, and endothelial repair.

137. A method for forming a targeted collagen product lacking an undesired characteristic, comprising: subtracting peptides exhibiting the undesired characteristic from collagen to form the targeted collagen product.

138. The method of aspect 137 above, further comprising the step of digesting the collagen to derive the peptides exhibiting the undesired characteristic therefrom.

139. The method of aspect 138 above, further comprising the step of testing the peptides for the undesired characteristic.

140. The method of aspect 139 above, further comprising the step of isolating the peptides exhibiting the undesired characteristic.

141. The method of aspect 137 above, wherein the subtracting step includes subtracting the peptides exhibiting the undesired characteristic to a mixture to reconstitute collagen into the targeted collagen product.

142. The method of aspect 137 above, wherein the subtracting step includes subtracting the peptide exhibiting the undesired characteristic to intact collagen.

143. The method of any of the above aspects, further comprising adding CHP or CMP peptide for amplifying collagen, wherein the CHP or CMP peptide is synthesized.

144. The method of aspect 143, wherein the CHP or CMP peptide is selected from the group consisting of a single-stranded peptide, a double-stranded peptide and a triple-stranded peptide.

145. The method of aspect 144 wherein the CHP or CMP peptide is modified with a polymerization group or a reactive group, whereby it can attach to collagen or modified collagen.

### BRIEF DESCRIPTION OF THE DRAWINGS

Embodiments of the invention are further described but are in no way limited by the following drawings.

FIG. 1 is a flow chart of a method according to a first embodiment of the present invention;

FIG. 2 is a flow chart of a method according to a second embodiment of the present invention;

FIG. 3 is a flow chart of a method according to a third embodiment of the present invention;

FIG. 4 is a flow chart of a method according to a fourth embodiment of the present invention.

FIG. 5 is a flow chart of the method according to fifth embodiment of the present invention;

FIG. 6 is a schematic view of a multilayer collagen product according to an embodiment of the present invention;

FIG. 7 is a is a schematic view of a hybrid medical product including a substrate and a collagen layer according to an embodiment of the present invention;

FIG. 8 is a flow chart of a method according to a sixth embodiment of the present invention;

FIG. 9 is a flow chart of a method according to a seventh embodiment of the present invention;

FIG. 10 is a flow chart of a method according to an eighth embodiment of the present invention; and

FIG. 11 is a flow chart of a method according to a ninth embodiment of the present invention.

### DETAILED DESCRIPTION OF THE INVENTION

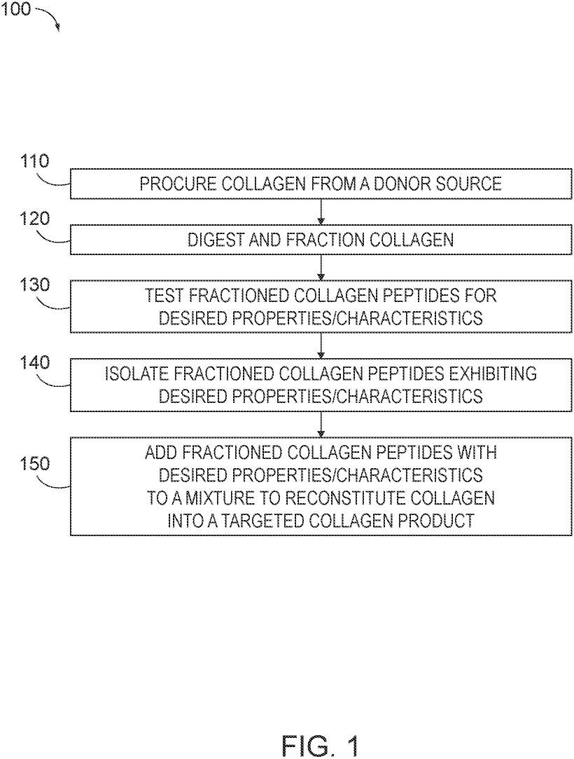

Reference is made to FIG. 1, which is a flowchart of a first exemplary embodiment of the method (100) according to the present disclosure, as further described below.

Collagen is procured (110) from an animal (e.g., bovine, porcine, equine, caprine) or a human source for processing, or genetically engineered from microorganisms or genetically engineered cells. Examples of collagen tissue that can be procured for this application are dermis, tendon, peritoneal tissue, pericardium, cartilage. Bone can also be a source of collagen to be further digested and fractionated. The type of collagen can be any type of collagen, for example, type 1, 3, etc. or genetically modified variants. Of course, it is also recognized that collagen can also be derived from genetically engineered microorganisms.

The collagen is then digested and fractioned (120), to break the collagen down into its constituent fragments, or peptides. Such peptides include, for example, P20 and P35. Commercial preparation is typically accomplished by one of five methods: (1) alkaline hydrolysis; (2) enzymatic hydrolysis (using e.g., pepsin, papain, collagenase, pancreatin, etc.); (3) acid hydrolysis; (4) a hybrid method of chemical/enzyme; (5) synthetically by fermentation. The hydrolyzed collagen is further fractionated by use of ultra-filtration membranes. In various embodiments, the peptides are synthesized on a peptide synthesizer.

Different peptides have different biological properties that imbue them with desired/desirable attributes/characteristics, such as a higher affinity for therapeutic usage in specific medical applications. In an embodiment, after digestion and fragmentation of the collagen tissue, each of the collagen fractions/peptides is tested (130) for various biological properties, such as, for example, attributes/characteristics that are useful for wound healing, blood clotting, bone formation/osteogenesis, cosmetic application, scaffolds for organ regeneration, use as antioxidants, antibacterial and/or anti-inflammatory activity, and for the delivery of drugs such as insulin and methylene blue, showing lower water absorbency.

In various embodiments, the collagen is tested by being subjected to an assay. Such an assay may include, for example, the assay of whole collagens in biological samples using a novel fluorogenic reagent, 3,4-dihydroxyphenylacetic acid (3,4-DHPAA), as described in H. Yasmin et al., Amplified and selective assay of collagens by enzymatic and fluorescent reactions, Scientific Reports, 4: 4950, May 13, 2014, which is incorporated by reference herein in its entirety. Cellular assays can be used to determine cellular activity, for example osteogenesis, to determine stimulatory activity, osseoinduction or osseoconduction. Another assay could be clotting ability of peptide via a blood assay for wound healing.

In some embodiments of the method, testing (130) of the collagen fractions/peptides is conducted only once. In other embodiments of the method, such testing may be conducted more than once. In still other embodiments no testing is conducted, e.g., where the biological properties of a collagen fraction/peptide are already known.

Following the performance of an assay or other testing, and based on the results thereof, or if already known from prior testing, collagen fractions/peptides having a specific desired/desirable property, such as, for example, facilitating wound healing of soft tissue, are identified, isolated (140) and selected for adding to a reconstituted collagen product. First, a native source of collagen, such as skin, bone, tendon, or ligament is cleaned, washed, and non-collagenous impurities removed by methods well known in the art (see, e.g., U.S. Pat. No. 5,512,291 and Oneson, et al., J. Am. Leather Chemists Assoc. 65:440-450, 1970, both of which are incorporated by reference herein in their entireties). The fibers obtained from the purification process are then further processed into a reconstituted collagen matrix. The reconstituted collagen matrix can be formed using the following general steps: a) forming an aqueous dispersion containing biopolymeric fibers; b) reconstituting the fibers; c) orienting the reconstituted fibers on a rotating mandrel to form a tubular membrane or forming them into a flat sheet if fabricating a sheet; d) compressing the hydrated fibers to remove excess solution to desired density or thickness; e) drying the fibers; and f) crosslinking the membrane. (see, e.g., U.S. Pat. Nos. 6,391,333, 6,599,524, and 7,807,192, all of which are incorporated by reference herein in their entireties).

The selected collagen fractions/peptides are added (150) to a mixture to reconstitute collagen into a “targeted collagen” having the amplified desired characteristic of the isolated fractions/peptides, such as a collagen targeted for soft tissue wound healing. In various embodiments, the collagen fractions or synthesized collagen peptide (e.g., CHP or CMP) can be a single-, double- or triple-stranded peptide.

In one embodiment, the selected fractioned, bioactive peptide(s) are added prior to performing the reconstituted collagen process, and cross-linked into the reconstituted collagen during the process. An alternative to adding the bioactive peptides to the reconstituted collagen would be adding such peptides after processing of the reconstituted collagen so that the bioactive peptides are not covalently linked into the collagen but are able to release, from the reconstituted collagen, over time. In other alternate embodiments, the bio-active peptide(s) are covalently linked to the reconstituted collagen after the collagen is reconstituted. In another embodiment, a bioactive peptide could be removed prior to being reconstituted so that the collagen does not have that bioactive characteristic. In another embodiment, the fraction alone is reconstituted into a medical device. Combinations and variations of these steps are also possible.

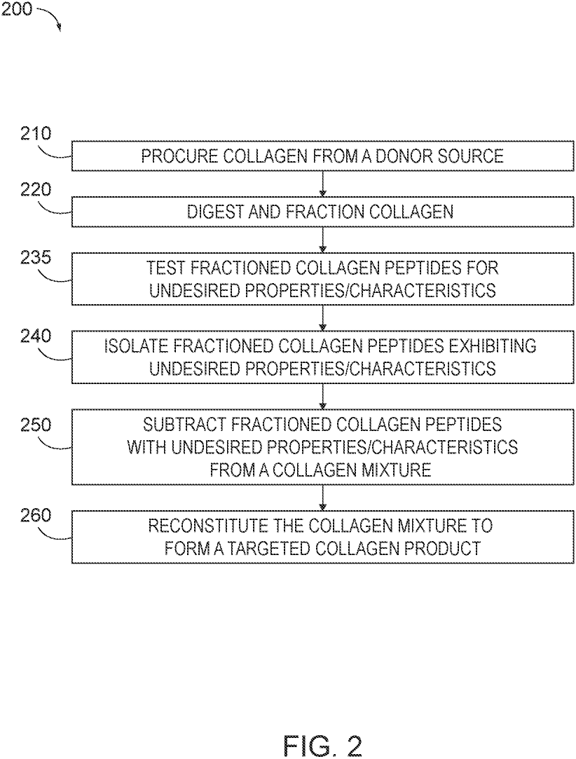

FIG. 2 illustrates an embodiment of the method (200) wherein a bioactive peptide is removed prior to being reconstituted so that the collagen does not have that bioactive characteristic. By removing a section of the collagen, the remaining collagen may have a bioactive peptide sequence, thereby actually concentrating that bioactive sequence after reconstituting. The method includes several of the same steps of the method (100) shown in FIG. 1 and discussed above, including procuring collagen (210) and digesting and fractioning the collagen (220) to yield peptides. The fractioned collagen peptides are then tested (235) for undesired properties/characteristics, and peptides having such undesired properties/characteristics are then isolated (240) and subtracted from a collagen mixture (255). The collagen mixture is then reconstituted (260) to form the targeted collagen product. In an alternate embodiment, the collagen mixture is not reconstituted, and the targeted collagen product is formed upon completion of the subtracting step (255).

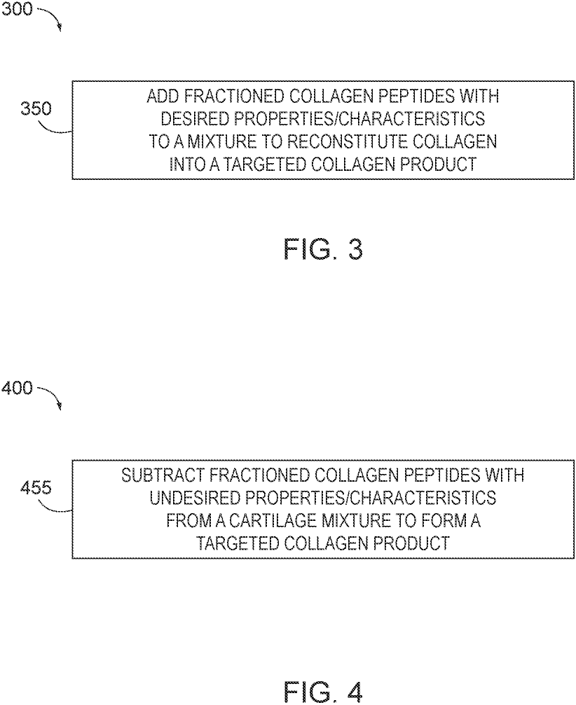

FIG. 3 illustrates another embodiment of the method (300) that includes the step (350) of adding fractioned collagen peptides with desired properties/characteristics to a mixture to reconstitute collagen into a targeted collagen product. In various embodiments, the method (300) may include additional steps, such as those illustrated in FIG. 1 and discussed above in connection with its associated method (100).

FIG. 4 illustrates still another embodiment of the method (400) that includes the step (455) of subtracting fractioned collagen peptides with undesired properties/characteristics from a mixture to form a targeted collagen product. In various embodiments, the method (400) may include additional steps, such as those illustrated in FIG. 2 and discussed above in connection with its associated method (200).

Also disclosed herein are collagen layers and surface coatings (the term “layers” being used herein to include both layers and surface coatings), wherein the collagen has been imbued with selective characteristics for various medical applications (as discussed in the embodiments above). Also disclosed herein are multi-layer collagen products, layered collagen/substrate products and methods for forming same.

In various embodiments of the disclosed invention, collagen is layered such that each layer contains reconstructed collagen that is different in properties and/or amino-acid content. The layers could contain more or less amino acid sequences that become useful at different stages of a biological process; wound healing process or bone repair process. The layers could contain amino acid sequences that allow collagen fibers to reconstitute at faster or slower rates when processing so that each layer has a distinct amount of collagen fiber so that each layer has different physical properties or biological activities.

In various embodiments, the layers of collagen are impregnated with active pharmaceutical ingredients (APIs), such as growth factors, bioactive peptides, Steroids, Antibiotics, Oncology, GI, Cardiovascular, Renal, AntiVirals, RNA(s), CNS, Neuromuscular and the like APIs which can be released into or onto the diseased or damaged part of a patient’s area following application or implantation.

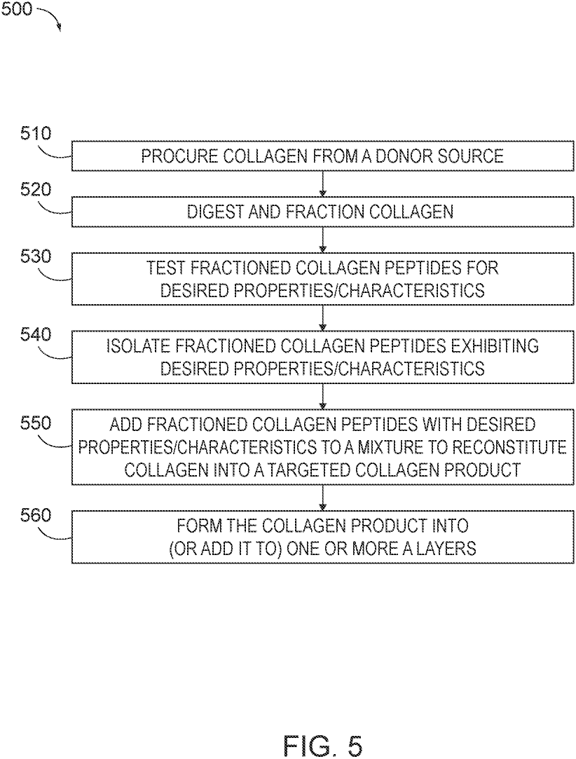

Reference is now made to FIG. 5, which is a flowchart of another exemplary embodiment of a method (500) according to the present disclosure, as further described below. The method (500) includes several of the same steps of the method (100) shown in FIG. 1 and discussed above, including procuring collagen (510), digesting and fractioning the collagen (520) to yield peptides, testing each of the collagen fractions/peptides (530) for various biological properties, isolating (140) the collagen fractions/peptides having a specific desired/desirable property, and adding the selected collagen fractions/peptides (550) to a mixture to reconstitute collagen into a “targeted collagen” having the amplified desired characteristic of the isolated fractions/peptides, such as a collagen targeted for soft tissue wound healing. Synthetically produced peptides (i.e., CHPs and/or CMPs) may alternatively be used in various embodiments.

In some embodiments of the method, the testing (530) of the collagen fractions/peptides is conducted only once. In other embodiments of the method, such testing may be conducted more than once. In still other embodiments no testing is conducted, e.g., where the biological properties of a collagen fraction/peptide are already known.

In one embodiment, the selected fractioned, bioactive peptide(s) are added prior to performing the reconstituted collagen process, and cross-linked into the reconstituted collagen during the process. An alternative to adding the bioactive peptides to the reconstituted collagen would be adding such peptides after processing of the reconstituted collagen so that the bioactive peptides are not covalently linked into the collagen but are able to release, from the reconstituted collagen, over time. In other alternate embodiments, the bioactive peptide(s) are covalently linked to the reconstituted collagen after the collagen is reconstituted. In other embodiments, the bioactive peptide(s) are added to collagen that has not been reconstituted. In another embodiment, a bioactive peptide could be removed prior to being reconstituted so that the collagen does not have that bioactive characteristic. In another embodiment, a portion of the collagen is removed to enhance the concentration of the remaining bioactive sites. This portion is then reconstituted to form a device. Combinations and variations of these steps are also possible.

In various embodiments, after the collagen product is formed according to the above steps (either reconstituted or not), it may be formed as or added to one or more layers (560). This layer(s) formation step or layering steps (560) enable the collagen amplification to be engineered in a layered way. In other words, the selected fractioned, bioactive peptide(s) is added to collagen to form the targeted collagen, which is then formed as one or more layers to facilitate control of a biologic process.

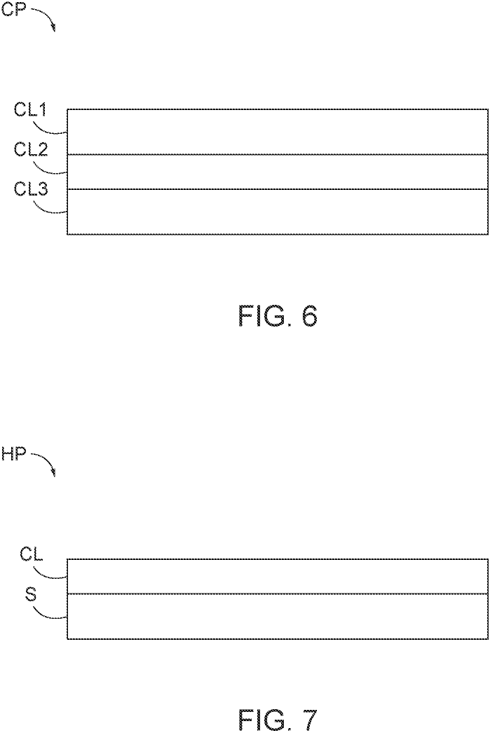

In some embodiments, two or more of the targeted collagen product layers are overlaid on each other to form a layered collagen product, such as a laminate. Reference is made to FIG. 6, which illustrates a layered collagen product CP having three collagen layers, CL1, CL2 and CL3. In various embodiments, the different targeted collagen layers CL1, CL2 and CL3 are engineered to have different bioactive properties. As a non-limiting example, the different collagen layers may be “programmed” to have different release times, e.g., for medications, proteins or other bioactive substance impregnated or selectively absorbed and released from its biological environment within one or more of the layers

In other embodiments, the targeted collagen layer is used to coat or contact a substrate or other surface that is not a targeted collagen layer. Reference is made to FIG. 7, which illustrates a layered hybrid product CP having a substrate S and a collagen layer CL. Whereas the collagen layer CL constitutes or includes the targeted collagen product formed according to the methods described herein, the substrate S may be untreated collagen, or any other biocompatible material. As a non-limiting example, the foregoing method can be used to form a collagen layer CL that has been amplified with a selected peptide that binds bone morphogenic proteins (BMPs) to cause osteoblast proliferation, and the substrate S is the outer surface of an orthopedic implant on which the collagen layer CL has been deposited In a further embodiment, a surface coating (not shown) is formed on the collagen layer CL opposite the implant surface/substrate S. The surface coating is designed to bind a high concentration of alpha2beta1 at the surface to recruit osteoblasts from a patient’s surrounding tissue. Once recruited onto the surface coating, the osteoblasts migrate into the underlying amplified collagen layer CL, the peptide of which binds BMP to cause the recruited osteoblasts to proliferate. In various embodiments, the substrate S includes collagen that has not been modified according to the methods disclosed herein (i.e., unmodified collagen).

In other embodiments of the method described above, a bioactive peptide is removed from (as opposed to added to) the collagen, so that the resulting collagen product does not have that bioactive characteristic. The collagen may be reconstituted or not reconstituted.

Targeted collagen product containing the specific collagen fractions/peptides is thus designed for specific medical applications, and provided in or formed as a collagen layer. For example, targeted collagen product layers may be engineered in this manner to address blood clotting, bone formation/osteogenesis, breast repair or wound healing. A programmed multilayer collagen product can be created with different specific collagen fractions/peptides in the different collagen layers, such that the layers have different bioactive properties and functions, as described above.

Further, it is recognized that a layered targeted collagen product may be formed from the process descried above, as well as any one or more additional processes known in the art.

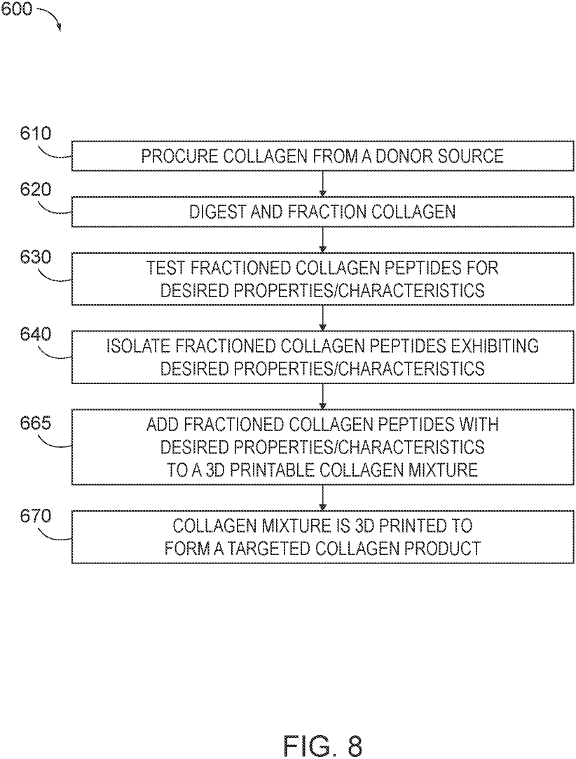

Reference is now made to FIG. 8, which is a flowchart of another exemplary embodiment of a method (600) according to the present disclosure, as further described below. The method (600) includes several of the same steps of the method (100) shown in FIG. 1 and discussed above, including procuring collagen (610), digesting and fractioning the collagen (620) to yield peptides, testing each of the collagen fractions/peptides (630) for various biological properties, and isolating (640) the collagen fractions/peptides having a specific desired/desirable property.

In some embodiments of the method, testing (630) of the collagen fractions/peptides is conducted only once. In other embodiments of the method, such testing may be conducted more than once. In still other embodiments no testing is conducted, e.g., where the biological properties of a collagen fraction/peptide are already known.

Known collagen mimic peptides (CMPs), single, double and triple helical CMPs, can be synthetically made via a peptide synthesis. Such CMPs can also be added to the reconstituted collagen matrix in various embodiments.

The selected collagen fractions/peptides are added (665) to a 3D printable collagen mixture, and the collagen mixture is then 3D printed (670) to form a “targeted collagen” having the amplified desired characteristic of the isolated fractions/peptides, such as a collagen targeted for soft tissue wound healing.

In one embodiment, the selected fractioned, bioactive peptide(s) are added prior to performing the 3D printing collagen process, and cross-linked into the collagen during the printing process. One alternative to adding the bioactive peptides to the printed collagen is adding such peptides after processing of the printed collagen so that the bioactive peptides are not covalently linked into the collagen but are able to release, from the reconstituted collagen, over time. In other alternate embodiments, the bio-active peptide(s) are covalently linked to the printed collagen after or while the collagen is printed. In another embodiment, a bioactive peptide could be removed prior to being printed so that the collagen does not have that bioactive characteristic. Combinations and variations of these steps are also possible.

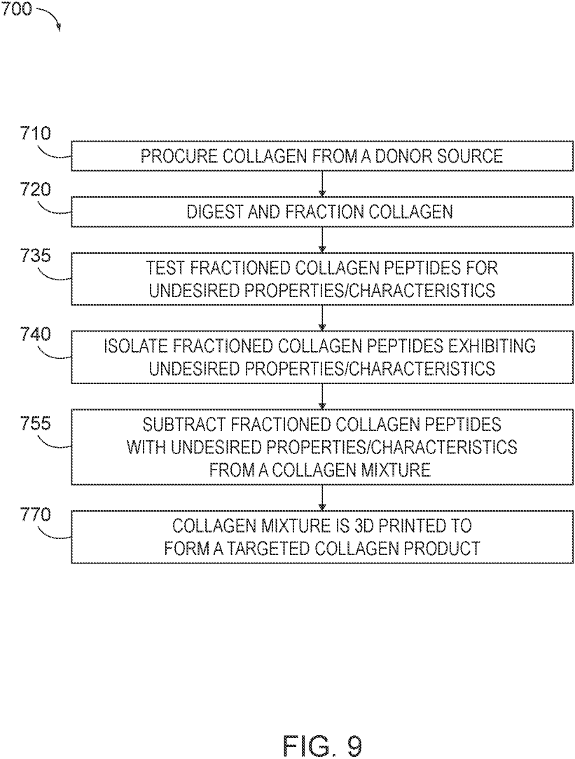

FIG. 9 illustrates another embodiment of a method (700) wherein a bioactive peptide is removed prior to being 3D printed so that the collagen does not have that bioactive characteristic. The method includes several of the same steps of the method (200) shown in FIG. 2 and discussed above, including procuring collagen (710), digesting and fractioning the collagen (720) to yield peptides, testing each of the collagen fractions/peptides (735) for various undesired biological properties, isolating (740) the collagen fractions/peptides having the undesired property, and subtracting (755) the collagen fractions/peptides having the undesired property from the collagen mixture. The collagen mixture is then 3D printed (770) to form the targeted collagen product.

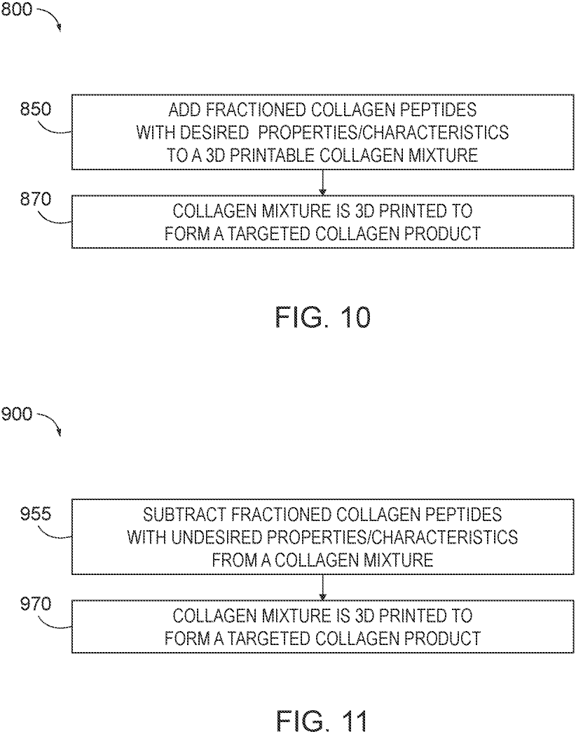

FIG. 10 illustrates another embodiment of a method (800) that includes the step (850) of adding fractioned collagen peptides with desired properties/characteristics to a mixture. The collagen mixture is then 3D printed (870) to form a targeted collagen product. In various embodiments, the method (800) may include additional steps, such as those illustrated in FIG. 8 and discussed above in connection with its associated method (600).

FIG. 11 illustrates still another embodiment of a method (900) that includes the step (955) of subtracting fractioned collagen peptides with undesired properties/characteristics from a mixture to form a targeted collagen product. The collagen mixture is then 3D printed (970) to form the targeted collagen product. In various embodiments, the method (900) may include additional steps, such as those illustrated in FIG. 9 and discussed above in connection with its associated method (700).

In various embodiments, the bioactive peptide(s), once determined, can be added to or subtracted from the collagen via genetic modification. The DNA or RNA can be modified via various CRISPR technologies, as well as zinc-finger nucleases (ZFN), or transcription activator-like endonucleases (TALENS). Once the modified collagen is synthesized within the cell, the modified collagen can be isolated via conventional means. In various embodiments, a bioactive peptide is removed from (rather than added to) the genetic modification.

Targeted collagen product containing the specific collagen fractions/peptides is thus designed for specific medical applications. For example, targeted collagen product may be engineered in this manner to address blood clotting, bone formation/osteogenesis, breast repair, or wound healing.

In some embodiments, the peptides with the desired characteristic can also be isolated and can also be added to or subtracted from intact collagen. For example, the blood clotting characteristic may be added or concentrated from a target collagen product if such product is intended to be used for a hemostasis product. The collagen is intact in an embodiment.

In various embodiments, the amplification peptide can be covalently, ionically, hydrogen bonded or through hydrophobic association linked into the collagen and/or allowed to release over time from the collagen. In another embodiment, the amplification peptide can be formulated with a biodegradable carrier so that it can release over time.

In other embodiments, the amplification peptide is added to or subtracted from the reconstituted collagen so that it impacts the pharmacokinetics or release characteristics of a bioactive protein. The collagen can also be genetically modified to impact the release of a bioactive protein or peptides. Examples of bioactive proteins that can be added are BMPs, PDGF, BDNF, EGF, VEGF, NGF, TNF and the like.

In some embodiments, the targeted collagen with the amplified peptide can be produced though genetically engineered prokaryotes and eukaryotes. This collagen is then further isolated via processing.

Of course, it is recognized that more than a single desired characteristic may be engineered into or out of the targeted collagen. For example, in skin wound healing, amplification of the collagen via bio-active peptides could be added for clotting, epithelial growth and faster resorption. A clotting test could identify the peptide that induces this process, an epithelial cell assay could identify the bioactive peptide for cell activity and the peptide that is targeted by the enzyme collagenase could be added to increase the collagen absorption.

Further, it is recognized that a targeted collagen product may be formed from the process descried above, as well as any one or more additional processes known in the art.

In general, any combination of disclosed features, components and methods described herein is possible. Steps of a method can be performed in any order that is physically possible.

All cited references are incorporated by reference herein.

Although embodiments have been disclosed, the invention is not limited thereby.

We claim:

1. A method for forming a targeted collagen product having an amplified desired characteristic, comprising: adding peptides exhibiting a desired characteristic to collagen to form the targeted collagen product.

2. The method of claim 1, further comprising the step of digesting the collagen to derive the peptides exhibiting the desired characteristic therefrom.

3. The method of claim 1, further comprising the step of procuring the collagen for the digesting step, and wherein the procuring step includes deriving collagen from the group consisting of eukaryotes, genetically modified prokaryotes and genetically modified plant cells.

4. The method of claim 2, further comprising the step of testing the peptides for the desired characteristic.

5. The method of claim 4, further comprising the step of isolating the peptides exhibiting the desired characteristic.

6. The method of claim 1, wherein the adding step includes adding the peptides exhibiting the desired characteristic to a mixture to reconstitute collagen into the targeted collagen product.

7. The method of claim 6, wherein the adding step is performed so that the reconstituted collagen can control the release profile of the peptide exhibiting the desired characteristic.

8. The method of claim 6, wherein the adding step is performed so that the reconstituted collagen is crosslinked to the peptide exhibiting the desired characteristic.

9. The method of claim 1, wherein the adding step includes adding the peptides exhibiting the desired characteristic to intact collagen.

10. The method of claim 1 wherein the desired characteristic of the peptides has an application selected from the group consisting of wound healing, osteogenesis, blood clotting, organ scaffolds, dura repair, nerve repair, ocular repair, epithelial repair, and cardio-vascular repair, breast repair, and endothelial repair.

11. A method for forming a targeted collagen product lacking an undesired characteristic, comprising: subtracting peptides exhibiting the undesired characteristic from collagen to form the targeted collagen product.

12. The method of claim 11, further comprising the step of digesting the collagen to derive the peptides exhibiting the undesired characteristic therefrom.

13. The method of claim 12, further comprising the step of testing the peptides for the undesired characteristic.

14. The method of claim 13, further comprising the step of isolating the peptides exhibiting the undesired characteristic.

15. The method of claim 11, wherein the subtracting step includes subtracting the peptides exhibiting the undesired characteristic to a mixture to reconstitute collagen into the targeted collagen product.

16. The method of claim 11, wherein the subtracting step includes subtracting the peptides exhibiting the undesired characteristic and reconstituting the remaining collagen that contains a desired characteristic thereby concentrating that desired characteristic.

17. The method of claim 11, wherein the subtracting step includes subtracting the peptide exhibiting the undesired characteristic to intact collagen.

18. The method of claim 10, wherein the peptide facilitates the binding to the collagen of cell types desirable for the application.

19. The method of claim 10, wherein the cell binding to the collagen occurs through the peptide and complementary cell integrins selected from α1β1, α2β1, α10β1 and α11β1.

20. The method of claim 18, wherein the desirable cell types are selected from osteoblasts, fibroblasts, epithelial cells and stem cells.

21. The method of claim 18, wherein the peptide is selected from the group consisting of RGD, GFOGER, DGEA, SVVYGLR, KRSR, FHRRIKA, P15, P20, or P35.

22. The method of claim 11, wherein the undesired characteristic is promotion of excessive thrombosis, inflammation, or osteolysis.

23. The method of claim 11, wherein the peptide facilitates the binding of cell types which are undesirable above maximum threshold concentrations.

24. The method of claim 23, wherein the cell types are selected from inflammatory cells, osteoclasts, and platelets.

25. The method of claim 24, wherein the inflammatory cells are selected from neutrophils, monocytes and lymphocytes.
